(19) United States
(12) Patent Application Publication
(10) Pub. No.: US 2010/0197752 A1
(43) Pub. Date: Aug. 5, 2010

(54) UROCANIC ACID DERIVATIVES USEFUL FOR THE TREATMENT OF IMMUNE-RELATED AND INFLAMMATORY DISEASES

(76) Inventor: Arthur Kammeijer, Amsterdan (NL)

Correspondence Address: RENNER OTTO BOISSELLE & SKLAR, LLP, 1621 EUCLID AVENUE, NINETEENTH FLOOR, CLEVELAND, OH, 44115, US

(21) Appl. No.: 12/664,072

(22) PCT Filed: Jun. 11, 2008
(86) PCT No.: PCT/NL08/50367

§ 371 Date: Apr. 5, 2010

Related U.S. Application Data
(60) Provisional application No. 60/944,382, filed on Jun. 15, 2007.

(30) Foreign Application Priority Data
Jun. 11, 2007 (GB) 0711234.5
Sep. 21, 2007 (GB) 0718543.2

(51) Int. Cl. A61K 31/4164; C07D 233/64; A61P 17/06; A61P 37/00

(52) U.S. Cl. 514/400; 548/341.5

(57) ABSTRACT

The invention relates to derivatives of urocanic acid that have improved efficacy and/or tissue penetration properties. The invention further provides use of these derivatives in a medicament for modulating an immune-related disease in an individual. Imidazole derivative, or a salt thereof, selected from O I1 W—C—R1 H— (formula 1); o I1 W—C—R1 and HN N r<1 NH (formula 2); O O Ov ̂ W—C—R1 and HN N (formula 3);

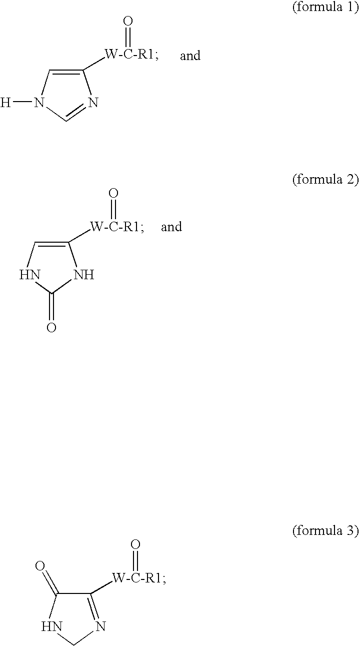

The invention relates to derivatives of urocanic acid that have improved efficacy and/or tissue penetration properties. The invention further provides use of these derivatives in a medicament for modulating an immune-related disease in an individual.

Ultraviolet radiation (UV), in particular the UVB range, is able to suppress the immune system. An explanation for the phenomenon of UV-mediated immunosuppression is that it prevents the recognition of molecules that are altered upon exposure to UV radiation as “non-self” neoantigens, which otherwise would result in chronically inflamed skin. However, a drawback of UV-mediated immunosuppression is that it enhances a risk of acquiring an infectious disease and of developing skin cancer.

Urocanic acid (UCA) is a major UV-absorbing chromophore in the epidermis and is one of the initiators of UV-induced immunosuppression. Trans-UCA is present in a non-exposed epidermis and can be photoisomerized by UV-exposure of the skin into cis-UCA (Norval et al. 1995. Photochem Photobiol. 62: 209-217; Noonan and De Fabo. 1002. Immunol Today 13: 250-254). In general, modulation or suppression of immune responses is provided by oxidation products of urocanic acid (UOPs), not cis-urocanic acid per se, comprising at least 3 UOPs: imidazole-4-carboxyaldehyde, imidazole-4-acetic acid or imidazole-4-carboxylic acid.

Imidazole-4-acetic acid (ImAc) can be formed from both trans- and cis-UCA isomers by photooxidation in the epidermis and in vitro (Kammeyer et al. 2001. Biochim. Biophys. Acta 1526: 277-285). ImAc has recently been shown to suppress the contact hypersensitivity (CHS) response in mice (Kammeyer et al. 2004. Photochem Photobiol. 80: 72-77), as was shown for cis-UCA by others (Norval et al. 1995. Photochem Photobiol. 62: 209-217; Noonan and De Fabo. 1992. Immunol Today 13: 250-254).

The present invention provides a new class of imidazole derivates with improved efficacy. The compounds are all imidazole derivatives, including imidazolones, with a modification at the C4 position of the imidazole ring, when compared to the imidazoles of WO 01/00145. The present invention therefore provides an imidazole derivative, or a salt thereof, selected from the group consisting of:

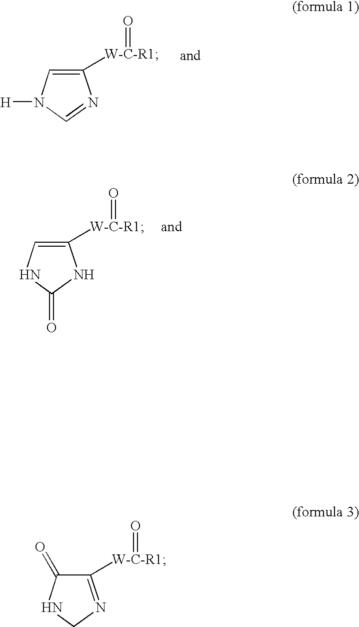

wherein:
W is either absent, or selected from (CH₂)y and CH═CH, wherein y=1 or 2; and
R1 is selected from —O—R2 and —N—(R3,R4), wherein R2 is a branched or unbranched, saturated or unsaturated, C₁-C₈ hydrocarbon chain; and wherein R3 and R4 independently represent hydrogen, or a branched or unbranched, saturated or unsaturated, C₁-C₈ hydrocarbon chain.

Imidazole derivatives, including imidazolone derivatives, of the invention show enhanced efficacy and/or tissue distribution upon administration, in particular when the imidazole derivative of the invention is compared with a derivative having the same backbone but a different R1 group, such as an oxidation product of urocanic acid. Furthermore, imidazole derivatives of the invention exhibit enhanced tissue penetration. Without being bound by theory, it is believed that the estimated pK_{o/w} (J. Garst, J. Pharm. Sci. 73 (1984) 1623-1629) of these imidazole derivatives is between zero and two, and that it is this property that enables enhanced efficacy and/or tissue penetration compared to ImAc and other oxidation products of urocanic acid. The imidazole derivatives of the invention more effectively reach and/or penetrate their cellular targets and show enhanced immunosuppressive behaviour. It is to be expected that longer hydrocarbon chains at the C4 position will increase the pKo/w value of the resulting compounds. Surprisingly, the modifications leave the immune suppressive quality of the compounds intact. Preferred imidazole derivatives, including imidazolone derivatives, according to the invention are presented in Table 1.

In particular preferred imidazole derivatives according to the invention are imidazole derivatives whereby said imidazole derivative comprises an imidazole ring structure according to formula 1. Imidazole derivatives comprising this imidazole ring structure have been identified as natural oxidation products of urocanic acid (UOPs) in the skin.

In one aspect of the invention, it is preferred that an imidazole derivative according to the invention is a compound according to formula 1, whereby W is absent.

Preferred examples of these imidazole derivatives are methyl imidazole-4-carboxylate, ethyl imidazole-4-carboxylate, propyl imidazole-4-carboxylate, isopropyl imidazole-4-carboxylate, butyl imidazole-4-carboxylate, sec butyl imidazole-4-carboxylate, tert butyl imidazole-4-carboxylate, pentyl imidazole-4-carboxylate, hexyl imidazole-4-carboxylate, heptyl imidazole-4-carboxylate, octyl imidazole-4-carboxylate, 2,3-dimethylpentyl imidazole-4-carboxylate, 2,3-dimethylpentyl imidazole-4-carboxylate, N-methyl imidazole-4-carboxylamide, N,N-dimethyl imidazole-4-carboxylamide, N-ethyl imidazole-4-carboxylamide, N,N-diethyl imidazole-4-carboxylamide, N-propyl imidazole-4-carboxylamide, N,N-dipropyl imidazole-4-carboxylamide, N-isopropyl imidazole-4-carboxylamide, N,N-diisopropyl imidazole-4-carboxylamide, N-butyl imidazole-4-carboxylamide, N,N-dibutyl imidazole-4-carboxylamide, N-sec-butyl imidazole-4-carboxylamide, N,N-di-sec-butyl imidazole-4-carboxylamide, N-tert-butyl imidazole-4-carboxylamide, N,N-di-tert-butyl imidazole-4-carboxylamide, N-pentyl imidazole-4-carboxylamide, N,N-dipentyl imidazole-4-carboxylamide, N-hexy imidazole-4-carboxylamide, N,N-dihexyl imidazole-4-carboxylamide, N-heptyl imidazole-4-carboxylamide, N,N-diheptyl imidazole-4-carboxylamide, N-octyl imidazole-4-carboxylamide, N,N-dioctyl imidazole-4-carboxylamide, N-(2,3-dimethylpentyl)imidazole-4-carboxylamide, N,N-di-(2,3-dimethylpentyl)imidazole-4-carboxylamide, N-(2,3-dimethylhexyl)imidazole-4-carboxylamide, and N,N-di-(2,3-dimethylhexyl)imidazole-4-carboxylamide.

A particularly preferred compound according to this aspect of the invention is ethyl imidazole-4-carboxylate. This compound has particularly advantageous efficacy, tissue penetration, tissue distribution and/or immune suppressive properties

In another aspect of the invention, a preferred imidazole derivative of the invention comprises a compound according to formula 1, whereby W is CH2.

Preferred examples of these imidazole derivatives according to this aspect of the invention are methyl imidazole-4-acetate, ethyl imidazole-4-acetate, propyl imidazole-4-acetate, isopropyl imidazole-4-acetate, butyl imidazole-4-acetate, sec butyl imidazole-4-acetate, tert butyl imidazole-4-acetate, pentyl imidazole-4-acetate, hexyl imidazole-4-acetate, heptyl imidazole-4-acetate, octyl imidazole-4-acetate, 2,3-dimethylpentyl imidazole-4-acetate, 2,3-dimethylpentyl imidazole-4-acetate, N-methyl imidazole-4-acetamide, N,N-dimethyl imidazole-4-acetamide, N-ethyl imidazole-4-acetamide, N,N-diethyl imidazole-4-acetamide, N-propyl imidazole-4-acetamide, N,N-dipropyl imidazole-4-acetamide, N-isopropyl imidazole-4-acetamide, N,N-diisopropyl imidazole-4-acetamide, N-butyl imidazole-4-acetamide, N,N-dibutyl imidazole-4-acetamide, N-sec-butyl imidazole-4-acetamide, N,N-di-sec-butyl imidazole-4-acetamide, N-tert-butyl imidazole-4-acetamide, N,N-di-tert-butyl imidazole-4-acetamide, N-pentyl imidazole-4-acetamide, N,N-dipentyl imidazole-4-acetamide, N-hexy imidazole-4-acetamide, N,N-dihexyl imidazole-4-acetamide, N-heptyl imidazole-4-acetamide, N,N-diheptyl imidazole-4-acetamide, N-octyl imidazole-4-acetamide, N,N-dioctyl imidazole-4-acetamide, and branched and/or saturated and unsaturated derivatives thereof such as N-(2,3-dimethylpentyl)imidazole-4-acetamide, N,N-di-(2,3-dimethylpentyl)imidazole-4-acetamide, N-(2,3-dimethylhexyl)imidazole-4-acetamide, and N,N-di-(2,3-dimethylhexyl)imidazole-4-acetamide.

A particularly preferred compound according to this aspect of the invention is ethyl imidazole-4-acetate. This compound has particularly advantageous efficacy, tissue penetration, tissue distribution and/or immune suppressive properties.

Without being bound by theory, it is to be expected that tissue penetration, distribution and immunosuppressive effects increase upon introduction of enlarged hydrocarbon chains from C₂ to C₈. Therefore, preferred compounds of the invention comprise compounds according to formula 1, whereby R1 is selected from —O—R2 and —N—(R3,R4), wherein R2, R3, and R4 are independently selected from a branched or unbranched, saturated or unsaturated, C₂-C₈ hydrocarbon chain, more preferred a branched or unbranched, saturated or unsaturated C₃-C₈ hydrocarbon chain, more preferred a branched or unbranched, saturated or unsaturated C4-C8 hydrocarbon chain, more preferred a branched or unbranched, saturated or unsaturated C₅-C₈ hydrocarbon chain, more preferred a branched or unbranched, saturated or unsaturated C₆-C₈ hydrocarbon chain, more preferred a branched or unbranched, saturated or unsaturated C₇-C₈ hydrocarbon chain, more preferred a branched or unbranched, saturated or unsaturated C₈ hydrocarbon chain.

Particularly preferred is an imidazole derivative according to formula 1, whereby R1 is selected from —O—R2 and —N—(R3,R4), wherein R2, R3, and R4 are independently selected from more preferred a branched or unbranched, saturated or unsaturated C4-C8 hydrocarbon chain.

Preferred imidazole derivatives of the invention are saturated, branched or unbranched, imidazole derivatives due to their improved skin penetration properties and increased pKo/w's over unsaturated chains. Unsaturated, branched or unbranched, imidazole derivatives, however, have an improved resistance to microbial degradation, compared to saturated imidazole derivatives. Therefore, unsaturated imidazole derivatives are preferred if enhanced stability of the imidazole derivatives is required.

In another aspect, the invention provides a use of an imidazole derivative according to the invention as a medicament.

The invention further provides the use of an imidazole derivative according to the invention in the preparation of a medicament for the treatment of an immune-related disease. The imidazole derivatives of the invention have anti-inflammatory properties and may thus be used as topical agents in dermatology, opthalmology and ear-nose-throat medicine. They may also be developed as systemic agents and then be used orally in a wide variety of inflammatory diseases.

An imidazole derivative of the invention was found to have immunosuppressive properties. Many immune related diseases are known, including immune-mediated inflammatory diseases, infectious diseases, immunodeficiency diseases, and cancer. Patients with immune related diseases that benefit from suppressing the immune response by an imidazole derivative of the invention, are patients suffering from especially immune-mediated and inflammatory diseases. Preferred examples of such immune-mediated and inflammatory diseases comprise systemic lupus erythematosis, arthritis, scleroderma, idiopathic inflammatory myopathies, including dermatomyositis and polymyositis, Crohn's disease, and dermatological diseases such as eczema, and psoriasis. A further beneficial application is suppression of the immune response in transplantations and degenerative neurological disorders like multiple sclerosis (MS) and amyotrophic lateral sclerosis (ALS).

A preferred use according to the invention is a medicament for the treatment of an immune-related or inflammatory dermatological disease, including but not limited to eczema and psoriasis.

Preferred examples of eczema that might be treated with a medicament of the invention comprise contact eczema such as allergic contact eczema and irritant contact eczema, perioral dermatitis, Poison Ivy dermatitis, dermatitis herpetiformis, Grover's disease; atopic eczema or atopiform eczema, discoid eczema, seborrhoeic eczema, and varicose eczema. Examples of psoriasis that might be treated with a medicament of the invention are plaque psoriasis, guttate psoriasis, inverse psoriasis, pustular psoriasis, erythrodermic psoriasis, scalp psoriasis, genital psoriasis, and psoriasis of the nails. Other preferred inflammatory diseases of the skin that can be treated with a compound and/or medicament of the invention include lupus erythematodes, lichen planus, and other popular and plaque type dermatological conditions.

In a further preferred embodiment, the invention provides a composition comprising an imidazole derivative according to the invention and carrier, diluent or excipient therefore.

A typical carrier for an imidazole derivative of the invention is an aqueous carrier such as water, and including a buffered aqueous solution comprising but not limited to phosphate buffered saline, and an aqueous alcoholic solution. An auxiliary agent such as a detergent can be added to the aqueous carrier to enhance the solubility of an imidazole derivative of the invention.

A typical diluent or excipient for an imidazole derivative of the invention comprises a binder such as starch or a cellulose derivative. Said diluent may also comprise a colored additive or a flavor enhancer.

The invention further provides a pharmaceutical composition comprising an imidazole derivative according to the invention and a pharmaceutically acceptable carrier, diluent or excipient therefore.

In formulating said imidazole derivative the imidazole derivative is adjusted to an appropriate concentration and formulated in a pharmaceutically and/or veterinarally acceptable carrier, diluent, excipient. Typical pharmaceutically acceptable carriers are known in the art and comprise phosphate buffered saline, oil including but not limited to mineral and vegetal oil, and aqueous solutions of, for example, sodium caroboxymethyl cellulose, magnesium stearate and polyvinylppyrrolidone.

The invention further provides the use of the pharmaceutical composition according to the invention for suppressing an immune response from an individual.

In a preferred embodiment, a pharmaceutical composition comprising an imidazole derivative of the invention is applied onto the skin. Said pharmaceutical composition can be an ointment, paste, cream, lotion, liquid, aerosol (spray), film or laminate, comprising said imidazole derivative.

Thus in a further aspect the invention provides an ointment, paste, cream, lotion, liquid, aerosol (spray), film and/or laminate, comprising an imidazole derivative of the invention.

In a preferred embodiment, said pharmaceutical composition further comprises other ingredients, such as beeswax, zinc oxide, allantoin, and/or, vitamin A, vitamin D and vitamin E, which may help to protect the skin.

In a preferred embodiment, said pharmaceutical composition further comprises solvents such as alcohol and propylene glycol, which are known to increase the solubility of, drugs in the skin layers, and may function as a penetration enhancer for transdermal therapeutic systems. Other penetration enhancers that are known in the art can be added to said pharmaceutical composition, including but not limited to laurocapram, methol, and vitamin E.

In a further preferred embodiment, said pharmaceutical composition further comprises ingredients that enhance the immune-suppressing activity of said imidazole derivative. Suitable immune suppressor enhancers comprise corticosteroids, methotrexate, azathioprine, cyclophosphamide, chlorambucil cyclosporine and tacrolimus and derivatives thereof such as rapamycin.

In a particularly preferred embodiment, said pharmaceutical composition further comprises corticosteroids. Suitable corticosteroids comprise prednisone, prednisolone, methylprednisolone, cortisone, hydrocortisone, fludrocortisone, dexamethasone, triamcinolone, budesonide and betamethasone.

The invention also provides a method of modulating an immune response in an individual in need of such treatment, said method comprising treating said individual with an effective amount of a pharmaceutical composition according to the invention.

The term “effective amount” refers to a concentration or amount of an imidazole derivative which results in achieving a particular stated purpose. An “effective amount” of an imidazole derivative may be determined empirically.

The invention further provides the use of an imidazole derivative, or a salt thereof of the invention for stimulating IL-10 production by hemopoietic cells. Preferably said cells are hemopoietic cells of the skin of blood cells. Preferably said imidazole derivative is ImCOOH, ImAc or Et-ImAc. In a particularly preferred embodiment said imidazole derivative is Et-ImAc.

### EXAMPLES

### Example 1

### Synthesis of ethyl imidazole-4-acetate

### Compounds:

Imidazole-4-acetic acid (ImAc) was synthesized by SynCom sample code 42583) and supplied by Chemshop, Weert.

Acetyl chloride was derived from Fluka (puriss.) as a colorless liquid.

Ethanol absolute (Lichrosolve, purity (>99.9% by GC) was purchased from VWR/Merck (nr. 1.00983)

### Procedure:

ImAc (6.3 g, 50 mmol) was dissolved in 80 ml ethanol. Acetyl chloride (11 g, 140 mmol, 10 ml) was dropwise added through a dripping funnel. The mixture was allowed to react for 5 h. During the first 30 minutes solid (starting) material completely dissolved. After cooling, the ethanol was evaporated on a Rotavapor™-device until ˜20 ml was left. Acidity was neutralized to pH=5 with sodium bicarbonate 8.4% and to ˜8 with NaOH 1 M or Na₂CO₃ 10%. Ethyl acetate (3×15 ml) was used to extract the waterphase under vigorously stirring. The EtOAc layer was separated with a separation funnel and the combined fractions were dried over anhydrous Na₂SO₄ while the solution was stirred. After 2 hours EtOAc was evaporated on a Rotavapor™-device. The crude product (˜6 g) was a yellow oily liquid that was treated for 3 hours in a Speedvac-device, set to 60° C.

The final product was referred to as: ImCH₂COOEt batch 2. (=Et-ImAc)

### Product Specifications

Yield: 5.94 g M.p.: <room temp. Appearance: brownish clear oil.

IR:

UV: λₘₐₓ=211 nm (H₂O), no abs. beyond 240 nm.

Mass: M/Z=154 confirmed.

HPLC: RP-column: Phenomenex Aqua 250×4.6 mm, eluent: ammonium formate 20 mM pH 5.1, CH₃CN 5%, D: 226 nm, F: 0.8 ml/min. R_{T} of product 11.98 min., 61807 AU/nmol. 0.2% imidazole-4-acetic acid as detected impurity.

### Example 2

Suppression of prolonged contact hypersensitivity (P-CHS) by ethyl imidazole-4-acetate in BALB/c mice.

### Topical Test Compounds.

Ethyl imidazole-4-acetate was dissolved in ethanol/water 1:1 in a concentration of 5%. The topical application of ethanol/water 1:1 alone served as full-response control. Ethanol/water test solutions were topically applied with a pipette in aliquots of 20 μL/ear.

### Prolonged Contact Hypersensitivity.

Mice were sensitized with 10 μl 1% oxazolone in acetone on day 6 in all experiments on the outside of both ears. On day 0 mice were challenged with 10 μl 0.5% oxazolone in acetone on both ears. Repeated elicitations were applied on day 2, 4, 7, 9. Applied oxazolone concentrations in acetone were 0.5%, 0.25%, 0.25%, 0.25%, respectively. From day 1 up to one day before the final day of the experiment, twice-daily doses of test solutions were topically given at approximately 11 AM and 16 PM. Duplicate ear thickness measurements were made on day 0, 1, 3, 5, 8, 10 and 11 prior to any daily topical application.

### Mice.

The use of mice was allowed by the Committee of Experimental Animal handling of the Academic Medical Center Amsterdam. Female BALB/c mice (8-10 weeks of age) were purchased from Charles River (L′Arbresle, France) and kept in light, humidity and temperature-controlled rooms in the animal facility, 1-2 weeks before the experiment. They were fed ad libitum with water and CRM-E food van Special Diets services (SDS, Witham, Essex, UK).

### Statistical Analyses

Data points, obtained with test compound administration, were compared to the data, obtained with vehicle (ethanol/water 1:1) administration and were statistically processed using Welch's unpaired t-test.

### Results

The averaged suppressive effects of imidazole-4-carboxylic acid, sodium salt (ImCOO.Na), Et-ImAc and prednisolone on P-CHS response, derived from 2-3 experiments are shown in FIG. 3 and compared to placebo (100% ear swelling; straight line). A comparison between the compounds can be made and a sequence from high to low effectiveness can be assigned as follows: prednisolone>ethyl imidazole-4-acetate (Et-ImAc)>imidazole-4-acetic acid (ImAc)=imidazole-4-carboxylic acid.

Therefore, we conclude that Et-ImAc is a new immunosuppressant. The immunosuppressive, e.g. anti-inflammatory properties of Et-ImAc are stronger than that of ImAc. This is likely due to the improved skin penetration of Et-ImAc, by which a similar molecular entity may reach immune target cells in higher concentrations than by topical application of ImAc. The efficacy seems to be between that of ImAc, a weak to moderate immunosuppressant, and prednisolone, a classical strong suppressant, but might require further optimization.

### Example 3

Effect of imidazole-derivates on the production of IL-10

IL-10 is a cytokine that dims inflammatory immune responses. ImCOOH, ImAc and Et-ImAc at a concentration of 10−⁴ Mol/l in whole blood results in the upregulation of IL-10 following Lipopolysaccharide (LPS) stimulation (10 ng/ml). The effect of Histamine as a positive control was also established and showed to exhibit a stronger effect on IL-10 production than the imidazole-derivates. Upregulation of IL-10 by ImCOOH, ImAc and Et-ImAc is expected to have favourable effects on disease activities of eczema, psoriasis and other inflammatory symptoms. These findings in vitro have larger significance in a complete test system as whole blood, employed here, than in a monoculture test system (FIG. 4).

### BRIEF DESCRIPTION OF THE DRAWING

FIG. 1. Effect of ImCH2COOEt of batch 1 on the relative ear swelling upon repeated elicitations on day 2, 4, 7, 9. From day 1 up to one day before the final day of the experiment, twice-daily doses of test solutions were topically given at approximately 11 AM and 16 PM.

FIG. 2. Effect of ImCH2COOEt of batch 2 on the relative ear swelling upon repeated elicitations on day 2, 4, 7, 9. From day 1 up to one day before the final day of the experiment, twice-daily doses of test solutions were topically given at approximately 11 AM and 16 PM.

FIG. 3. The averaged suppressive effects of imidazole-4-carboxylic acid, sodium salt (ImCOO.Na), Et-ImAc and prednisolone on P-CHS response, derived from 2-3 experiments are shown in FIG. 3 and compared to placebo (100% ear swelling; straight line).

FIG. 4. Effect of imidazole-derivates on the production of IL-10 in whole blood

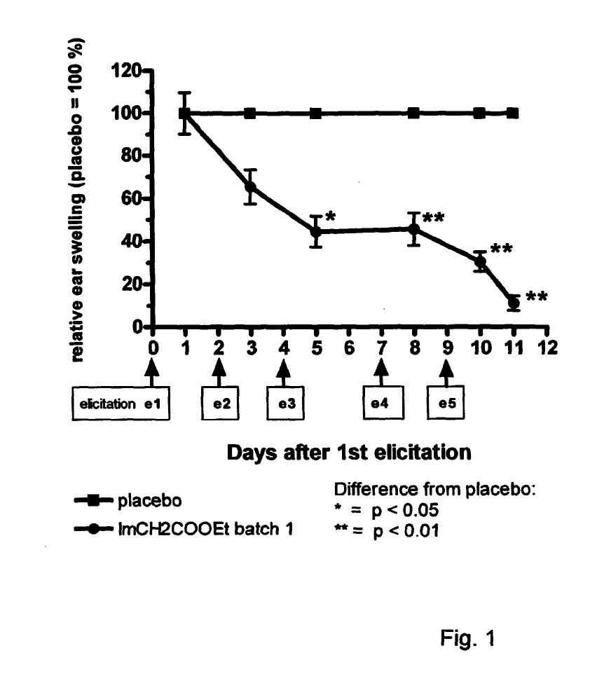

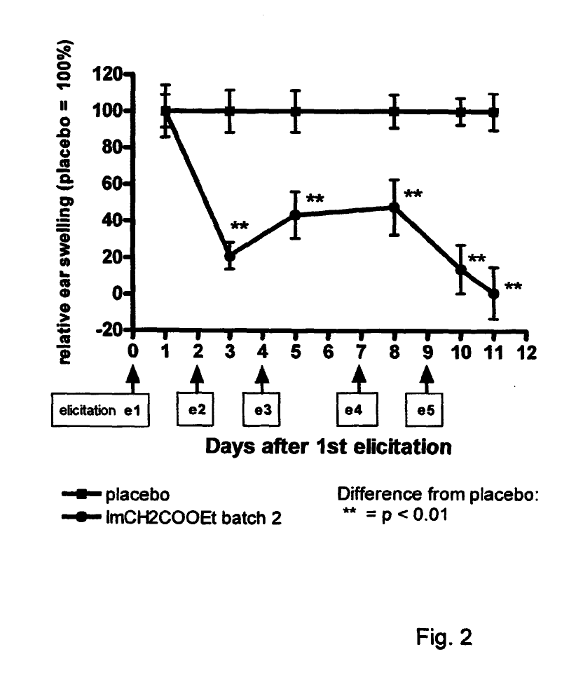

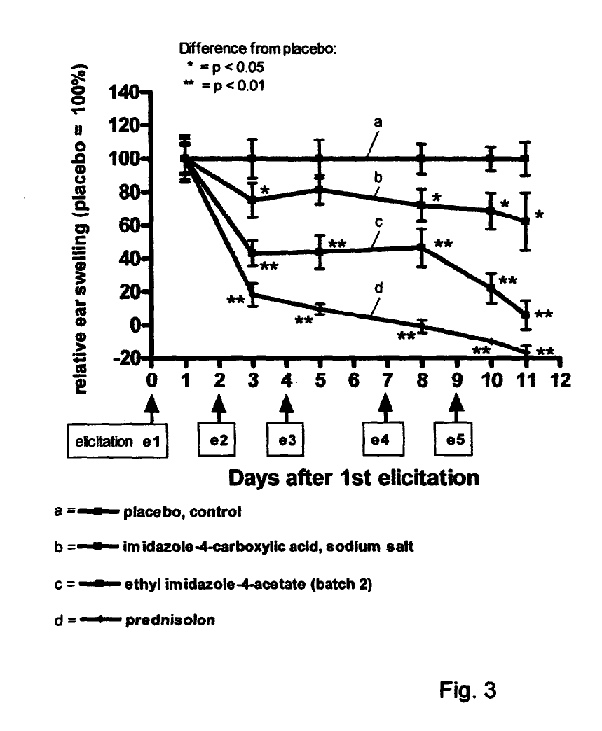

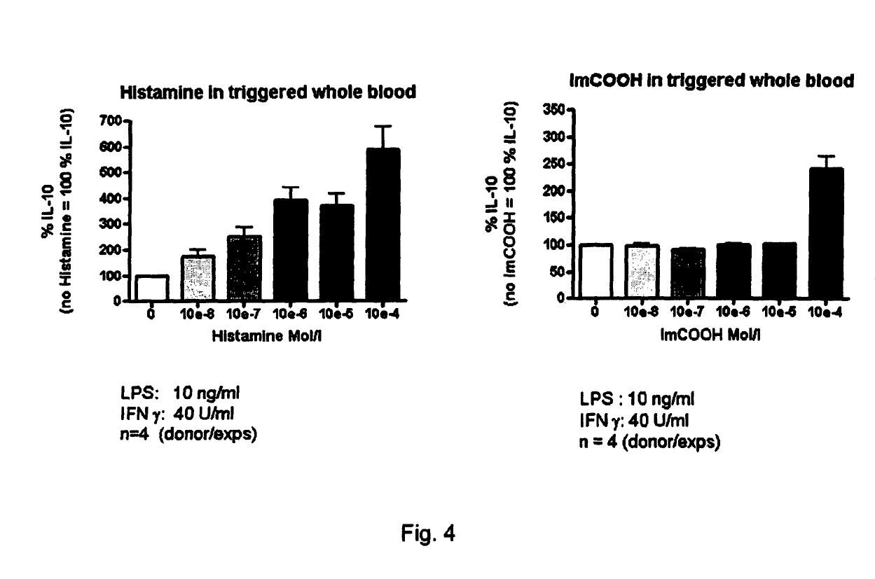

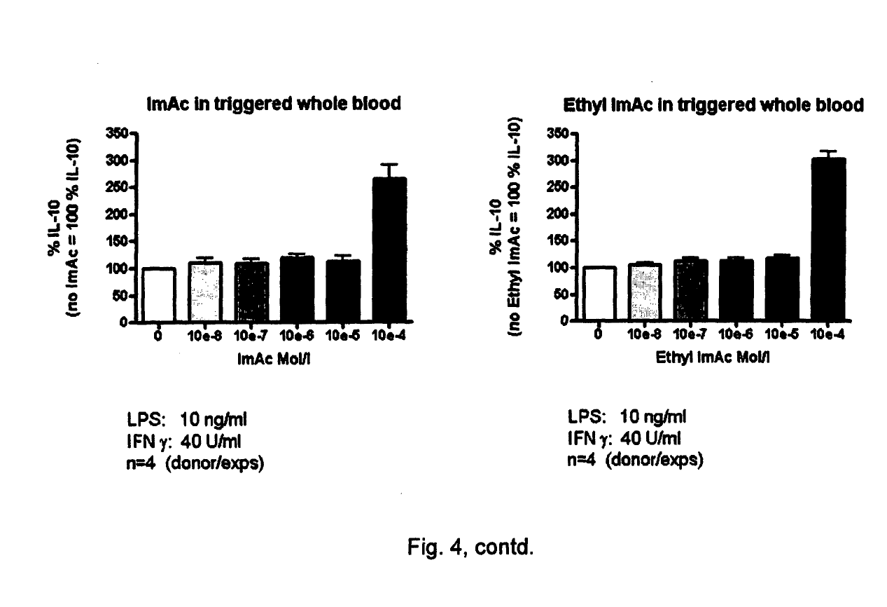

TABLE 1

| | | | |
|---|---|---|---|
| ethyl imidazole-4-acetate | ethyl imidazole-4-carboxylate | ethyl imidazole-4-propionic acid | ethyl imidazole-4-propenoic acid |
| propyl imidazole-4-acetate | propyl imidazole-4-carboxylate | propyl imidazole-4-propionic acid | propyl imidazole-4-propenoic acid |
| isopropyl imidazole-4-acetate | isopropyl imidazole-4-carboxylate | isopropyl imidazole-4-propionic | isopropyl imidazole-4-propenoic acid |
| | | acid | |
| butyl imidazole-4-acetate | butyl imidazole-4-carboxylate | butyl imidazole-4-propionic acid | butyl imidazole-4-propenoic acid |
| sec butyl imidazole-4-acetate | sec butyl imidazole-4-carboxylate | sec butyl imidazole-4-propionic | sec butyl imidazole-4-propenoic acid |
| | | acid | |
| tert butyl imidazole-4-acetate | tert butyl imidazole-4-carboxylate | tert butyl imidazole-4-propionic | tert butyl imidazole-4-propenoic acid |
| | | acid | |
| pentyl imidazole-4-acetate | pentyl imidazole-4-carboxylate | pentyl imidazole-4-propionic acid | pentyl imidazole-4-propenoic acid |
| hexyl imidazole-4-acetate | hexyl imidazole-4-carboxylate | hexyl imidazole-4-propionic acid | hexyl imidazole-4-propenoic acid |
| heptyl imidazole-4-acetate | heptyl imidazole-4-carboxylate | heptyl imidazole-4-propionic acid | heptyl imidazole-4-propenoic acid |
| octyl imidazole-4-acetate | octyl imidazole-4-carboxylate | octyl imidazole-4-propionic acid | octyl imidazole-4-propenoic acid |
| 2,3-dimethylpentyl imidazole-4- | 2,3-dimethylpentyl imidazole-4- | 2,3-dimethylpentyl imidazole-4- | 2,3-dimethylpentyl imidazole-4-propenoic |
| acetate | carboxylate | propionic acid | acid |
| 2,3-dimethylpentyl imidazole-4- | 2,3-dimethylpentyl imidazole-4- | 2,3-dimethylpentyl imidazole-4- | 2,3-dimethylpentyl imidazole-4-propenoic |
| acetate | carboxylate | propionic acid | acid |
| N-methyl imidazole-4-acetamide | N-methyl imidazole-4- | N-methyl imidazole-4- | N-methyl imidazole-4-propenoylamide |
| | carboxylamide | propionylamide | |
| N,N-dimethyl imidazole-4-acetamide | N,N-dimethyl imidazole-4- | N,N-dimethyl imidazole-4- | N,N-dimethyl imidazole-4-propenoylamide |
| | carboxylamide | propionylamide | |
| N-ethyl imidazole-4-acetamide | N-ethyl imidazole-4-carboxylamide | N-ethyl imidazole-4- | N-ethyl imidazole-4-propenoylamide |
| | | propionylamide | |
| N,N-diethyl imidazole-4-acetamide | N,N-diethyl imidazole-4- | N,N-diethyl imidazole-4- | N,N-diethyl imidazole-4-propenoylamide |
| | carboxylamide | propionylamide | |
| N-propyl imidazole-4-acetamide | N-propyl imidazole-4-carboxylamide | N-propyl imidazole-4- | N-propyl imidazole-4-propenoylamide |
| | | propionylamide | |
| N,N-dipropyl imidazole-4-acetamide | N,N-dipropyl imidazole-4- | N,N-dipropyl imidazole-4- | N,N-dipropyl imidazole-4-propenoylamide |
| | carboxylamide | propionylamide | |
| N-isopropyl imidazole-4-acetamide | N-isopropyl imidazole-4- | N-isopropyl imidazole-4- | N-isopropyl imidazole-4-propenoylamide |
| | carboxylamide | propionylamide | |
| N,N-diisopropyl imidazole-4- | N,N-diisopropyl imidazole-4- | N,N-diisopropyl imidazole-4- | N,N-diisopropyl imidazole-4- |
| acetamide | carboxylamide | propionylamide | propenoylamide |
| N-butyl imidazole-4-acetamide | N-butyl imidazole-4-carboxylamide | N-butyl imidazole-4- | N-butyl imidazole-4-propenoylamide |
| | | propionylamide | |
| N,N-dibutyl imidazole-4-acetamide | N,N-dibutyl imidazole-4- | N,N-dibutyl imidazole-4- | N,N-dibutyl imidazole-4-propenoylamide |
| | carboxylamide | propionylamide | |
| N-sec-butyl imidazole-4-acetamide | N-sec-butyl imidazole-4- | N-sec-butyl imidazole-4- | N-sec-butyl imidazole-4-propenoylamide |
| | carboxylamide | propionylamide | |
| N,N-di-sec-butyl imidazole-4- | N,N-di-sec-butyl imidazole-4- | N,N-di-sec-butyl imidazole-4- | N,N-di-sec-butyl imidazole-4- |
| acetamide | carboxylamide | propionylamide | propenoylamide |
| N-tert-butyl imidazole-4-acetamide | N-tert-butyl imidazole-4- | N-tert-butyl imidazole-4- | N-tert-butyl imidazole-4-propenoylamide |
| | carboxylamide | propionylamide | |
| N,N-di-tert-butyl imidazole-4- | N,N-di-tert-butyl imidazole-4- | N,N-di-tert-butyl imidazole-4- | N,N-di-tert-butyl imidazole-4- |
| acetamide | carboxylamide | propionylamide | propenoylamide |
| N-pentyl imidazole-4-acetamide | N-pentyl imidazole-4-carboxylamide | N-pentyl imidazole-4- | N-pentyl imidazole-4-propenoylamide |
| | | propionylamide | |
| N,N-dipentyl imidazole-4-acetamide | N,N-dipentyl imidazole-4- | N,N-dipentyl imidazole-4- | N,N-dipentyl imidazole-4-propenoylamide |
| | carboxylamide | propionylamide | |
| N-hexy imidazole-4-acetamide | N-hexy imidazole-4-carboxylamide | N-hexy imidazole-4- | N-hexy imidazole-4-propenoylamide |
| | | propionylamide | |
| N,N-dihexyl imidazole-4-acetamide | N,N-dihexyl imidazole-4- | N,N-dihexyl imidazole-4- | N,N-dihexylimidazole-4-propenoylamide |
| | carboxylamide | propionylamide | |
| N-heptyl imidazole-4-acetamide | N-heptyl imidazole-4-carboxylamide | N-heptyl imidazole-4- | N-heptyl imidazole-4-propenoylamide |
| | | propionylamide | |
| N,N-diheptyl imidazole-4-acetamide | N,N-diheptyl imidazole-4- | N,N-diheptyl imidazole-4- | N,N-diheptyl imidazole-4-propenoylamide |
| | carboxylamide | propionylamide | |
| N-octyl imidazole-4-acetamide | N-octyl imidazole-4-carboxylamide | N-octyl imidazole-4- | N-octyl imidazole-4-propenoylamide |
| | | propionylamide | |
| N,N-dioctyl imidazole-4-acetamide | N,N-dioctyl imidazole-4- | N,N-dioctyl imidazole-4- | N,N-dioctyl imidazole-4-propenoylamide |
| | carboxylamide | propionylamide | |
| N-(2,3-dimethylpentyl)imidazole-4- | N-(2,3-dimethylpentyl)imidazole-4- | N-(2,3-dimethylpentyl)imidazole- | N-(2,3-dimethylpentyl)imidazole-4- |
| acetamide | carboxylamide | 4-propionylamide | propenoylamide |
| N,N-di-(2,3- | N,N-di-(2,3- | N,N-di-(2,3- | N,N-di-(2,3-dimethylpentyl)imidazole-4- |
| dimethylpentyl)imidazole-4- | dimethylpentyl)imidazole-4- | dimethylpentyl)imidazole-4- | propenoylamide |
| acetamide | carboxylamide | propionylamide | |
| N-(2,3-dimethylhexyl)imidazole-4- | N-(2,3-dimethylhexyl)imidazole-4- | N-(2,3-dimethylhexyl)imidazole- | N-(2,3-dimethylhexyl)imidazole-4- |
| acetamide | carboxylamide | 4-propionylamide | propenoylamide |
| N,N-di-(2,3-dimethylhexyl)imidazole- | N,N-di-(2,3- | N,N-di-(2,3- | N,N-di-(2,3-dimethylhexyl)imidazole-4- |
| 4-acetamide | dimethylhexyl)imidazole-4- | dimethylhexyl)imidazole-4- | propenoylamide |
| | carboxylamide | propionylamide | |
| ethyl imidazol-2-one-4-acetate | ethyl imidazol-2-one-4-carboxylate | ethyl imidazol-2-one-4-propionic | ethyl imidazol-2-one-4-propenoic acid |
| | | acid | |
| propyl imidazol-2-one-4-acetate | propyl imidazol-2-one-4-carboxylate | propyl imidazol-2-one-4-propionic | propyl imidazol-2-one-4-propenoic acid |
| | | acid | |
| isopropyl imidazol-2-one-4-acetate | isopropyl imidazol-2-one-4- | isopropyl imidazol-2-one-4- | isopropyl imidazol-2-one-4-propenoic acid |
| | carboxylate | propionic acid | |
| butyl imidazol-2-one-4-acetate | butyl imidazol-2-one-4-carboxylate | butyl imidazol-2-one-4-propionic | butyl imidazol-2-one-4-propenoic acid |
| | | acid | |
| sec butyl imidazol-2-one-4-acetate | sec butyl imidazol-2-one-4- | sec butyl imidazol-2-one-4- | sec butyl imidazol-2-one-4-propenoic acid |
| | carboxylate | propionic acid | |
| tert butyl imidazol-2-one-4-acetate | tert butyl imidazol-2-one-4- | tert butyl imidazol-2-one-4- | tert butyl imidazol-2-one-4-propenoic acid |
| | carboxylate | propionic acid | |
| pentyl imidazol-2-one-4-acetate | pentyl imidazol-2-one-4-carboxylate | pentyl imidazol-2-one-4-propionic | pentyl imidazol-2-one-4-propenoic acid |
| | | acid | |
| hexyl imidazol-2-one-4-acetate | hexyl imidazol-2-one-4-carboxylate | hexyl imidazol-2-one-4-propionic | hexyl imidazol-2-one-4-propenoic acid |
| | | acid | |
| heptyl imidazol-2-one-4-acetate | heptyl imidazol-2-one-4-carboxylate | heptyl imidazol-2-one-4-propionic | heptyl imidazol-2-one-4-propenoic acid |
| | | acid | |
| octyl imidazol-2-one-4-acetate | octyl imidazol-2-one-4-carboxylate | octyl imidazol-2-one-4-propionic | octyl imidazol-2-one-4-propenoic acid |
| | | acid | |
| 2,3-dimethylpentyl imidazol-2-one-4- | 2,3-dimethylpentyl imidazol-2-one- | 2,3-dimethylpentyl imidazol-2- | 2,3-dimethylpentyl imidazol-2-one-4- |
| acetate | 4-carboxylate | one-4-propionic acid | propenoic acid |
| 2,3-dimethylpentyl imidazol-2-one-4- | 2,3-dimethylpentyl imidazol-2-one- | 2,3-dimethylpentyl imidazol-2- | 2,3-dimethylpentyl imidazol-2-one-4- |
| acetate | 4-carboxylate | one-4-propionic acid | propenoic acid |
| N-methyl imidazol-2-one-4-acetamide | N-methyl imidazol-2-one-4- | N-methyl imidazol-2-one-4- | N-methyl imidazol-2-one-4-propenoylamide |
| | carboxylamide | propionylamide | |
| N,N-dimethyl imidazol-2-one-4- | N,N-dimethyl imidazol-2-one-4- | N,N-dimethyl imidazol-2-one-4- | N,N-dimethyl imidazol-2-one-4- |
| acetamide | carboxylamide | propionylamide | propenoylamide |
| N-ethyl imidazol-2-one-4-acetamide | N-ethyl imidazol-2-one-4- | N-ethyl imidazol-2-one-4- | N-ethyl imidazol-2-one-4-propenoylamide |
| | carboxylamide | propionylamide | |
| N,N-diethyl imidazol-2-one-4- | N,N-diethyl imidazol-2-one-4- | N,N-diethyl imidazol-2-one-4- | N,N-diethyl imidazol-2-one-4- |
| acetamide | carboxylamide | propionylamide | propenoylamide |
| N-propyl imidazol-2-one-4-acetamide | N-propyl imidazol-2-one-4- | N-propyl imidazol-2-one-4- | N-propyl imidazol-2-one-4-propenoylamide |
| | carboxylamide | propionylamide | |
| N,N-dipropyl imidazol-2-one-4- | N,N-dipropyl imidazol-2-one-4- | N,N-dipropyl imidazol-2-one-4- | N,N-dipropyl imidazol-2-one-4- |
| acetamide | carboxylamide | propionylamide | propenoylamide |
| N-isopropyl imidazol-2-one-4- | N-isopropyl imidazol-2-one-4- | N-isopropyl imidazol-2-one-4- | N-isopropyl imidazol-2-one-4- |
| acetamide | carboxylamide | propionylamide | propenoylamide |
| N,N-diisopropyl imidazol-2-one-4- | N,N-diisopropyl imidazol-2-one-4- | N,N-diisopropyl imidazol-2-one-4- | N,N-diisopropyl imidazol-2-one-4- |
| acetamide | carboxylamide | propionylamide | propenoylamide |
| N-butyl imidazol-2-one-4-acetamide | N-butyl imidazol-2-one-4- | N-butyl imidazol-2-one-4- | N-butyl imidazol-2-one-4-propenoylamide |
| | carboxylamide | propionylamide | |
| N,N-dibutyl imidazol-2-one-4- | N,N-dibutyl imidazol-2-one-4- | N,N-dibutyl imidazol-2-one-4- | N,N-dibutyl imidazol-2-one-4- |
| acetamide | carboxylamide | propionylamide | propenoylamide |
| N-sec-butyl imidazol-2-one-4- | N-sec-butyl imidazol-2-one-4- | N-sec-butyl imidazol-2-one-4- | N-sec-butyl imidazol-2-one-4- |
| acetamide | carboxylamide | propionylamide | propenoylamide |
| N,N-di-sec-butyl imidazol-2-one-4- | N,N-di-sec-butyl imidazol-2-one-4- | N,N-di-sec-butyl imidazol-2-one- | N,N-di-sec-butyl imidazol-2-one-4- |
| acetamide | carboxylamide | 4-propionylamide | propenoylamide |
| N-tert-butyl imidazol-2-one-4- | N-tert-butyl imidazol-2-one-4- | N-tert-butyl imidazol-2-one-4- | N-tert-butyl imidazol-2-one-4- |
| acetamide | carboxylamide | propionylamide | propenoylamide |
| N,N-di-tert-butyl imidazol-2-one-4- | N,N-di-tert-butyl imidazol-2-one-4- | N,N-di-tert-butyl imidazol-2-one- | N,N-di-tert-butyl imidazol-2-one-4- |
| acetamide | carboxylamide | 4-propionylamide | propenoylamide |
| N-pentyl imidazol-2-one-4-acetamide | N-pentyl imidazol-2-one-4- | N-pentyl imidazol-2-one-4- | N-pentyl imidazol-2-one-4-propenoylamide |
| | carboxylamide | propionylamide | |
| N,N-dipentyl imidazol-2-one-4- | N,N-dipentyl imidazol-2-one-4- | N,N-dipentyl imidazol-2-one-4- | N,N-dipentyl imidazol-2-one-4- |
| acetamide | carboxylamide | propionylamide | propenoylamide |
| N-hexy imidazol-2-one-4-acetamide | N-hexy imidazol-2-one-4- | N-hexy imidazol-2-one-4- | N-hexy imidazol-2-one-4-propenoylamide |
| | carboxylamide | propionylamide | |
| N,N-dihexyl imidazol-2-one-4- | N,N-dihexyl imidazol-2-one-4- | N,N-dihexyl imidazol-2-one-4- | N,N-dihexyl imidazol-2-one-4- |
| acetamide | carboxylamide | propionylamide | propenoylamide |
| N-heptyl imidazol-2-one-4-acetamide | N-heptyl imidazol-2-one-4- | N-heptyl imidazol-2-one-4- | N-heptyl imidazol-2-one-4-propenoylamide |
| | carboxylamide | propionylamide | |
| N,N-diheptyl imidazol-2-one-4- | N,N-diheptyl imidazol-2-one-4- | N,N-diheptyl imidazol-2-one-4- | N,N-diheptyl imidazol-2-one-4- |
| acetamide | carboxylamide | propionylamide | propenoylamide |
| N-octyl imidazol-2-one-4-acetamide | N-octyl imidazol-2-one-4- | N-octyl imidazol-2-one-4- | N-octyl imidazol-2-one-4-propenoylamide |
| | carboxylamide | propionylamide | |
| N,N-dioctyl imidazol-2-one-4- | N,N-dioctyl imidazol-2-one-4- | N,N-dioctyl imidazol-2-one-4- | N,N-dioctyl imidazol-2-one-4- |
| acetamide | carboxylamide | propionylamide | propenoylamide |
| N-(2,3-dimethylpentyl)imidazol-2- | N-(2,3-dimethylpentyl)imidazol-2- | N-(2,3-dimethylpentyl)imidazol- | N-(2,3-dimethylpentyl)imidazol-2-one-4- |
| one-4-acetamide | one-4-carboxylamide | 2-one-4-propionylamide | propenoylamide |
| N,N-di-(2,3-dimethylpentyl)imidazol- | N,N-di-(2,3- | N,N-di-(2,3- | N,N-di-(2,3-dimethylpentyl)imidazol-2-one- |
| 2-one-4-acetamide | dimethylpentyl)imidazol-2-one-4- | dimethylpentyl)imidazol-2-one-4- | 4-propenoylamide |
| | carboxylamide | propionylamide | |
| N-(2,3-dimethylhexyl)imidazol-2- | N-(2,3-dimethylhexyl)imidazol-2- | N-(2,3-dimethylhexyl)imidazol-2- | N-(2,3-dimethylhexyl)imidazol-2-one-4- |
| one-4-acetamide | one-4-carboxylamide | one-4-propionylamide | propenoylamide |
| N,N-di-(2,3-dimethylhexyl)imidazol- | N,N-di-(2,3-dimethylhexyl)imidazol- | N,N-di-(2,3- | N,N-di-(2,3-dimethylhexyl)imidazol-2-one- |
| 2-one-4-acetamide | 2-one-4-carboxylamide | dimethylhexyl)imidazol-2-one-4- | 4-propenoylamide |
| | | propionylamide | |
| ethyl imidazol-5-one-4-acetate | ethyl imidazol-5-one-4-carboxylate | ethyl imidazol-5-one-4-propionic | ethyl imidazol-5-one-4-propenoic acid |
| | | acid | |
| propyl imidazol-5-one-4-acetate | propyl imidazol-5-one-4-carboxylate | propyl imidazol-5-one-4-propionic | propyl imidazol-5-one-4-propenoic acid |
| | | acid | |
| isopropyl imidazol-5-one-4-acetate | isopropyl imidazol-5-one-4- | isopropyl imidazol-5-one-4- | isopropyl imidazol-5-one-4-propenoic acid |
| | carboxylate | propionic acid | |
| butyl imidazol-5-one-4-acetate | butyl imidazol-5-one-4-carboxylate | butyl imidazol-5-one-4-propionic | butyl imidazol-5-one-4-propenoic acid |
| | | acid | |
| sec butyl imidazol-5-one-4-acetate | sec butyl imidazol-5-one-4- | sec butyl imidazol-5-one-4- | sec butyl imidazol-5-one-4-propenoic acid |
| | carboxylate | propionic acid | |
| tert butyl imidazol-5-one-4-acetate | tert butyl imidazol-5-one-4- | tert butyl imidazol-5-one-4- | tert butyl imidazol-5-one-4-propenoic acid |
| | carboxylate | propionic acid | |
| pentyl imidazol-5-one-4-acetate | pentyl imidazol-5-one-4-carboxylate | pentyl imidazol-5-one-4-propionic | pentyl imidazol-5-one-4-propenoic acid |
| | | acid | |
| hexyl imidazol-5-one-4-acetate | hexyl imidazol-5-one-4-carboxylate | hexyl imidazol-5-one-4-propionic | hexyl imidazol-5-one-4-propenoic acid |
| | | acid | |
| heptyl imidazol-5-one-4-acetate | heptyl imidazol-5-one-4-carboxylate | heptyl imidazol-5-one-4-propionic | heptyl imidazol-5-one-4-propenoic acid |
| | | acid | |
| octyl imidazol-5-one-4-acetate | octyl imidazol-5-one-4-carboxylate | octyl imidazol-5-one-4-propionic | octyl imidazol-5-one-4-propenoic acid |
| | | acid | |
| 2,3-dimethylpentyl imidazol-5-one-4- | 2,3-dimethylpentyl imidazol-5-one- | 2,3-dimethylpentyl imidazol-5- | 2,3-dimethylpentyl imidazol-5-one-4- |
| acetate | 4-carboxylate | one-4-propionic acid | propenoic acid |
| 2,3-dimethylpentyl imidazol-5-one-4- | 2,3-dimethylpentyl imidazol-5-one- | 2,3-dimethylpentyl imidazol-5- | 2,3-dimethylpentyl imidazol-5-one-4- |
| acetate | 4-carboxylate | one-4-propionic acid | propenoic acid |
| N-methyl imidazol-5-one-4-acetamide | N-methyl imidazol-5-one-4- | N-methyl imidazol-5-one-4- | N-methyl imidazol-5-one-4-propenoylamide |
| | carboxylamide | propionylamide | |
| N,N-dimethyl imidazol-5-one-4- | N,N-dimethyl imidazol-5-one-4- | N,N-dimethyl imidazol-5-one-4- | N,N-dimethyl imidazol-5-one-4- |
| acetamide | carboxylamide | propionylamide | propenoylamide |
| N-ethyl imidazol-5-one-4-acetamide | N-ethyl imidazol-5-one-4- | N-ethyl imidazol-5-one-4- | N-ethyl imidazol-5-one-4-propenoylamide |
| | carboxylamide | propionylamide | |
| N,N-diethyl imidazol-5-one-4- | N,N-diethyl imidazol-5-one-4- | N,N-diethyl imidazol-5-one-4- | N,N-diethyl imidazol-5-one-4- |
| acetamide | carboxylamide | propionylamide | propenoylamide |
| N-propyl imidazol-5-one-4-acetamide | N-propyl imidazol-5-one-4- | N-propyl imidazol-5-one-4- | N-propyl imidazol-5-one-4-propenoylamide |
| | carboxylamide | propionylamide | |
| N,N-dipropyl imidazol-5-one-4- | N,N-dipropyl imidazol-5-one-4- | N,N-dipropyl imidazol-5-one-4- | N,N-dipropyl imidazol-5-one-4- |
| acetamide | carboxylamide | propionylamide | propenoylamide |
| N-isopropyl imidazol-5-one-4- | N-isopropyl imidazol-5-one-4- | N-isopropyl imidazol-5-one-4- | N-isopropyl imidazol-5-one-4- |
| acetamide | carboxylamide | propionylamide | propenoylamide |
| N,N-diisopropyl imidazol-5-one-4- | N,N-diisopropyl imidazol-5-one-4- | N,N-diisopropyl imidazol-5-one-4- | N,N-diisopropyl imidazol-5-one-4- |
| acetamide | carboxylamide | propionylamide | propenoylamide |
| N-butyl imidazol-5-one-4-acetamide | N-butyl imidazol-5-one-4- | N-butyl imidazol-5-one-4- | N-butyl imidazol-5-one-4-propenoylamide |
| | carboxylamide | propionylamide | |
| N,N-dibutyl imidazol-5-one-4- | N,N-dibutyl imidazol-5-one-4- | N,N-dibutyl imidazol-5-one-4- | N,N-dibutyl imidazol-5-one-4- |
| acetamide | carboxylamide | propionylamide | propenoylamide |
| N-sec-butyl imidazol-5-one-4- | N-sec-butyl imidazol-5-one-4- | N-sec-butyl imidazol-5-one-4- | N-sec-butyl imidazol-5-one-4- |
| acetamide | carboxylamide | propionylamide | propenoylamide |
| N,N-di-sec-butyl imidazol-5-one-4- | N,N-di-sec-butyl imidazol-5-one-4- | N,N-di-sec-butyl imidazol-5-one- | N,N-di-sec-butyl imidazol-5-one-4- |
| acetamide | carboxylamide | 4-propionylamide | propenoylamide |
| N-tert-butyl imidazol-5-one-4- | N-tert-butyl imidazol-5-one-4- | N-tert-butyl imidazol-5-one-4- | N-tert-butyl imidazol-5-one-4- |
| acetamide | carboxylamide | propionylamide | propenoylamide |
| N,N-di-tert-butyl imidazol-5-one-4- | N,N-di-tert-butyl imidazol-5-one-4- | N,N-di-tert-butyl imidazol-5-one- | N,N-di-tert-butyl imidazol-5-one-4- |
| acetamide | carboxylamide | 4-propionylamide | propenoylamide |
| N-pentyl imidazol-5-one-4-acetamide | N-pentyl imidazol-5-one-4- | N-pentyl imidazol-5-one-4- | N-pentyl imidazol-5-one-4-propenoylamide |
| | carboxylamide | propionylamide | |
| N,N-dipentyl imidazol-5-one-4- | N,N-dipentyl imidazol-5-one-4- | N,N-dipentyl imidazol-5-one-4- | N,N-dipentyl imidazol-5-one-4- |
| acetamide | carboxylamide | propionylamide | propenoylamide |
| N-hexyl imidazol-5-one-4-acetamide | N-hexyl imidazol-5-one-4- | N-hexy imidazol-5-one-4- | N-hexy imidazol-5-one-4-propenoylamide |
| | carboxylamide | propionylamide | |
| N,N-dihexyl imidazol-5-one-4- | N,N-dihexyl imidazol-5-one-4- | N,N-dihexyl imidazol-5-one-4- | N,N-dihexyl imidazol-5-one-4- |
| acetamide | carboxylamide | propionylamide | propenoylamide |
| N-heptyl imidazol-5-one-4-acetamide | N-heptyl imidazol-5-one-4- | N-heptyl imidazol-5-one-4- | N-heptyl imidazol-5-one-4-propenoylamide |
| | carboxylamide | propionylamide | |
| N,N-diheptyl imidazol-5-one-4- | N,N-diheptyl imidazol-5-one-4- | N,N-diheptyl imidazol-5-one-4- | N,N-diheptyl imidazol-5-one-4- |
| acetamide | carboxylamide | propionylamide | propenoylamide |
| N-octyl imidazol-5-one-4-acetamide | N-octyl imidazol-5-one-4- | N-octyl imidazol-5-one-4- | N-octyl imidazol-5-one-4-propenoylamide |
| | carboxylamide | propionylamide | |
| N,N-dioctyl imidazol-5-one-4- | N,N-dioctyl imidazol-5-one-4- | N,N-dioctyl imidazol-5-one-4- | N,N-dioctyl imidazol-5-one-4- |
| acetamide | carboxylamide | propionylamide | propenoylamide |
| N-(2,3-dimethylpentyl)imidazol-5- | N-(2,3-dimethylpentyl)imidazol-5- | N-(2,3-dimethylpentyl)imidazol- | N-(2,3-dimethylpentyl)imidazol-5-one-4- |
| one-4-acetamide | one-4-carboxylamide | 5-one-4-propionylamide | propenoylamide |
| N,N-di-(2,3-dimethylpentyl)imidazol- | N,N-di-(2,3- | N,N-di-(2,3- | N,N-di-(2,3-dimethylpentyl)imidazol-5-one- |
| 5-one-4-acetamide | dimethylpentyl)imidazol-5-one-4- | dimethylpentyl)imidazol-5-one-4- | 4-propenoylamide |
| | carboxylamide | propionylamide | |
| N-(2,3-dimethylhexyl)imidazol-5- | N-(2,3-dimethylhexyl)imidazol-5- | N-(2,3-dimethylhexyl)imidazol-5- | N-(2,3-dimethylhexyl)imidazol-5-one-4- |
| one-4-acetamide | one-4-carboxylamide | one-4-propionylamide | propenoylamide |
| N,N-di-(2,3-dimethylhexyl)imidazol- | N,N-di-(2,3-dimethylhexyl)imidazol- | N,N-di-(2,3- | N,N-di-(2,3-dimethylhexyl)imidazol-5-one- |
| 5-one-4-acetamide | 5-one-4-carboxylamide | dimethylhexyl)imidazol-5-one-4- | 4-propenoylamide |
| | | propionylamide | |

1. Imidazole derivative, or a salt thereof, selected from 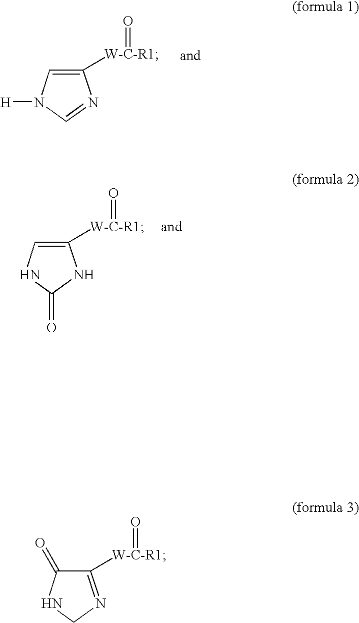
wherein:
W is either absent, or selected from (CH₂)y and CH═CH, wherein y=1 or 2; and
R1 is selected from —O—R2 and —N—(R3,R4), wherein R2 is a branched or unbranched, saturated or unsaturated C₁-C₈, hydrocarbon chain; and wherein R3 and R4 independently represent hydrogen, or a branched or unbranched, saturated or unsaturated C₁-C₈ hydrocarbon chain.

2. An imidazole derivative selected from the derivatives presented in Table 1.

3. Imidazole derivative according to claim 1, whereby said imidazole derivative comprises an imidazole ring structure according to formula 1.

4. Imidazole derivative according to claim 3, whereby W is absent.

5. Imidazole derivative according to claim 3, whereby W is CH₂.

6. Imidazole derivative according to claim 5, whereby said derivative is ethyl imidazole-4-acetate.

7. Imidazole derivative according to formula 1, whereby R2, R3, and R4 are independently selected from a branched or unbranched, saturated or unsaturated C4-C8 hydrocarbon chain.

8. Imidazole derivative according to claim 1, whereby said R2, R3, and R4 each comprises a saturated, branched or unbranched hydrocarbon chain.

9. Use of an imidazole derivative according to claim 1, or a pharmaceutically acceptable salt thereof, as a medicament.

10. Use of an imidazole derivative according to claim 1 in the preparation of a medicament for modulating an immune-related disease.

11. Use according to claim 9, whereby said immune-related disease is a dermatological disease.

12. Use according to claim 11, whereby said dermatological disease is psoriasis or eczema.

13. A composition comprising an imidazole derivative according to claim 1 and a carrier, diluent or excipient therefore.

14. A pharmaceutical composition comprising an imidazole derivative according to claim 1 and a pharmaceutically acceptable carrier, diluent or excipient therefore.

15. Use of the pharmaceutical composition of claim 14 for modulating an immune-related disease of an individual.

16. Method of modulating an immune-related disease of an individual in need of such treatment, said method comprising treating said individual with an effective amount of the pharmaceutical composition of claim 14
